(19) United States
(12) Patent Application Publication
(10) Pub. No.: US 2024/0016366 A1
(43) Pub. Date: Jan. 18, 2024

(54) IMAGE DIAGNOSIS SYSTEM FOR LESION

(71) Applicant: AIDOT INC., Seoul (KR)

(72) Inventor: Jae Hoon JEONG, Seongnam-si (KR)

(73) Assignee: AIDOT INC., Seoul (KR)

(21) Appl. No.: 18/038,649

(22) PCT Filed: Dec. 15, 2020
(86) PCT No.: PCT/KR2020/018312

§ 371 Date: Jul. 12, 2023

(30) Foreign Application Priority Data
Nov. 25, 2020 (KR) 10-2020-0159910

(51) Int. Cl. A61B 1/00; G16H 50/20; G06T 7/00; G16H 30/40

(52) U.S. Cl. CPC A61B 1/000096; A61B 1/00045; G16H 50/20; G06T 7/0012; G16H 30/40; G06T 2207/10068; G06T 2207/20081; G06T 2207/20084

(57) ABSTRACT

The present invention relates to a system for diagnosing a lesion on an endoscopic image, and includes an observation image acquisition unit configured to acquire an observation image from an input endoscopic image, a pre-processing unit configured to pre-process an acquired observation image, a lesion diagnosis unit configured to diagnose a degree of lesion on the pre-processed observation image using a pre-trained artificial neural network learning model for lesion diagnosis, and a screen display control unit configured to display and output a lesion diagnosis result.

### TECHNICAL FIELD

The present invention relates to a system for diagnosing an image lesion, and more particularly, to a system for diagnosing a lesion on an endoscopic image.

### BACKGROUND ART

An endoscope can be used to diagnose conditions inside the body or detect a lesion. As an endoscopic examination method for obtaining an image of the inside of the body, a method of taking a picture of the inside by inserting a flexible tube to which a camera is attached through a patient's mouth or anus into a digestive organ, etc. is widely used.

However, since a general endoscopic examination method has limitations in observing the inside of a narrow, long and complexly curved digestive organ such as the small intestine, a capsule type endoscope has been developed and used. The capsule type endoscope is a pill-shaped microscopic endoscope having a diameter of about 9 to 11 mm and a length of about 24 to 26 mm. In the capsule type endoscope, when a patient swallows it, just like a pill, the camera of the endoscope collects images of the inside of organs such as the stomach, small intestine, and large intestine and transmits them to an external receiver, and a diagnostician diagnoses an internal state of the organs while observing the internal body image transmitted by the capsule type endoscope through a display unit.

As such, since the diagnosis of the lesion on the endoscopic image is usually made by visual observation by a specialist, different diagnosis results can be obtained depending on the experience, ability, and proficiency of the specialist. Therefore, for the endoscopic image, a new objective and reliable diagnostic method is needed regardless of the experience, ability, and proficiency of the specialist

### PRIOR ART LITERATURE

### Patent Literature

(PTL 1) Korean unexamined patent application publication No. 10-2020-0070062
(PTL 2) Korean unexamined patent application publication No. 10-2020-0038121

### DISCLOSURE OF THE INVENTION

### Technical Problem

Therefore, the present invention is an invention devised in accordance with the needs described above, and a main object of the present invention is to provide a system for diagnosing an image lesion capable of automatically detecting and diagnosing a lesion on a photographed endoscopic image or an endoscopic image obtainable from endoscopic equipment.

Furthermore, another object of the present invention is to provide a system for diagnosing an image lesion capable of automatically diagnosing the lesion by acquiring only an image frozen in the endoscopic equipment.

In addition, another object of the present invention is to provide a system for diagnosing an image lesion capable of automatically diagnosing a lesion by acquiring only an image frozen in the endoscopic equipment, as well as detecting a lesion in real time from the endoscopic image acquired from the endoscopic equipment and diagnosing the lesion.

Furthermore, another object of the present invention is to provide a system for diagnosing an image lesion capable of automatically diagnosing the presence or absence of the lesion on the endoscopic image, and diagnosing and displaying a degree of lesion and a depth of infiltration.

Furthermore, another object of the present invention is to provide a system for diagnosing an image lesion constructed to diagnose a lesion not only on a gastric endoscopic image, but also on a small intestine endoscopic image and/or large intestine endoscopic image.

### Technical Solution

A system for diagnosing an image lesion according to an embodiment of the present invention for achieving the objects described above includes

an observation image acquisition unit configured to acquire an observation image from an input endoscopic image,

a pre-processing unit configured to pre-process an acquired observation image,

a lesion diagnosis unit configured to diagnose a degree of lesion on the pre-processed observation image using a pre-trained artificial neural network learning model for lesion diagnosis, and

a screen display control unit configured to display and output a lesion diagnosis result, and

the observation image acquisition unit acquires, as the observation image, image frames whose inter-frame similarity exceeds a predetermined threshold among frames of the endoscopic image.

In some cases, the observation image acquisition unit may capture and acquire the endoscopic image as an observation image when an electric signal generated according to a machine freeze operation of an endoscope equipment operator is input.

A system for diagnosing an image lesion according to another embodiment of the present invention includes

a pre-processing unit configured to pre-process an input endoscopic image,

a lesion area detection unit configured to detect a lesion area in real time from the pre-processed endoscopic image frame using a pre-trained artificial neural network learning model for real-time lesion area detection,

a screen display control unit configured to display and output an endoscopic image frame in which the detected lesion area is marked.

The lesion area detection unit of the system for diagnosing the image lesion including this configuration includes

a pre-trained artificial neural network learning model for detecting one or more lesion areas in order to detect a lesion area for each of one or more endoscopic images among a gastric endoscope image, a small intestine endoscopy image, and a large intestine endoscopy image.

A system for diagnosing an image lesion according to another embodiment of the present invention includes

a pre-processing unit configured to pre-process an input endoscopic image,

a lesion area detection unit configured to detect a lesion area in real time from the pre-processed endoscopic image frame using a pre-trained artificial neural network learning model for real-time lesion area detection,

a lesion diagnosis unit configured to diagnose a degree of lesion for the detected lesion area using a pre-trained artificial neural network learning model for lesion diagnosis, and

a screen display control unit configured to display and output the detected lesion area and a lesion diagnosis result.

### Advantageous Effects

According to the means for solving the technical problems described above, the system for diagnosing the image lesion according to an embodiment of the present invention has an advantage capable of diagnosing a lesion by automatically recognizing an image frozen by a machine in the endoscopic equipment and obtaining the image as an observation image, and

Furthermore, it has the advantage capable of obtaining objective and highly reliable diagnosis results regardless of the experience, ability, and proficiency of a specialist because the degree of lesion is automatically diagnosed using the pre-trained artificial neural network learning model for lesion diagnosis on the acquired observation image, that is, the image frozen in the endoscopic equipment and the result is displayed.

In addition, the present invention, by including a pre-trained artificial neural network learning model for one or more lesion areas detection and a pre-trained artificial neural network learning model for one or more lesion diagnoses in order to detect a lesion area for each of one or more endoscopic images among a gastric endoscope image, a small intestine endoscopy image, and a large intestine endoscopy image, has the advantage capable of automatically diagnosing the degree of lesion by automatically detecting the lesion area for a gastric endoscope, a large intestine endoscope, and a small intestine endoscope according to an operation mode (which is set to gastric endoscope diagnosis mode, . . . , internal endoscope diagnosis mode) with only one system construction, and

can be implemented as an embedded system in the endoscopic equipment so that the endoscopic equipment itself automatically diagnoses the lesion on the endoscope image.

### BRIEF DESCRIPTION OF THE DRAWINGS

FIG. 1 is an exemplary diagram illustrating a peripheral configuration of a system for diagnosing an image lesion according to an embodiment of the present invention.

FIGS. 2 to 4 are diagrams illustrating configurations of the system for diagnosing the image lesion according to embodiments of the present invention.

FIGS. 5 and 6 are operational flow diagrams of the system for diagnosing the image lesion according to embodiments of the present invention.

FIG. 7 is a diagram for describing observation image acquisition according to an embodiment of the present invention.

FIGS. 8A to 9B are exemplary diagrams for diagnosing the degrees of lesions on endoscopic images according to embodiments of the present invention.

FIGS. 10 and 11 are exemplary diagrams of lesion diagnosis screens according to an embodiment of the present invention.

### MODE FOR CARRYING OUT THE INVENTION

A detailed description of the present invention to be described later refers to the accompanying drawings, which illustrate specific embodiments in which the present invention may be practiced, in order to make the objects, technical solutions, and advantages of the present invention clear. These embodiments are described in detail sufficient to enable a person skilled in the art to practice the present invention.

Throughout the detailed description and claims of the present invention, ‘learning’ is a term referring to performing machine learning according to a procedure, and thus a person skilled in the art will understand that it is not intended to refer to a mental operation such as a human educational activity. In addition, throughout the detailed description and claims of the present invention, the word ‘include’ and its variants are not intended to exclude other technical features, additions, components, or steps. Other objects, advantages and characteristics of the present invention will be revealed to a person skilled in the art, in part from this description and in part from practice of the present invention. The examples and drawings below are provided as examples and are not intended to limit the present invention. Furthermore, the present invention covers all possible combinations of the embodiments shown in this specification. It should be understood that various embodiments of the present invention are different from each other but are not necessarily mutually exclusive. For example, the configurations and characteristics described herein may be implemented in other embodiments without departing from the spirit and scope of this invention in relation to one embodiment. In addition, it should be understood that the location or arrangement of individual components within each disclosed embodiment may be changed without departing from the spirit and scope of the present invention. Accordingly, the detailed description described later is not intended to be taken in a limiting sense, and the scope of the present invention, if properly described, is limited only by the appended claims, along with all ranges equivalent to those claimed by the claims. Similar reference numerals in the drawings indicate the same or similar functions in several aspects.

In this specification, unless otherwise indicated or clearly contradicted by context, terms referred to in the singular include the plural unless otherwise required in that context. In addition, in describing the present invention, if it is determined that a detailed description of a related known configuration or function may obscure the gist of the present invention, the detailed description thereof will be omitted.

Hereinafter, in order to enable a person skilled in the art to easily practice the present invention, preferred embodiments of the present invention will be described in detail with reference to the accompanying drawings.

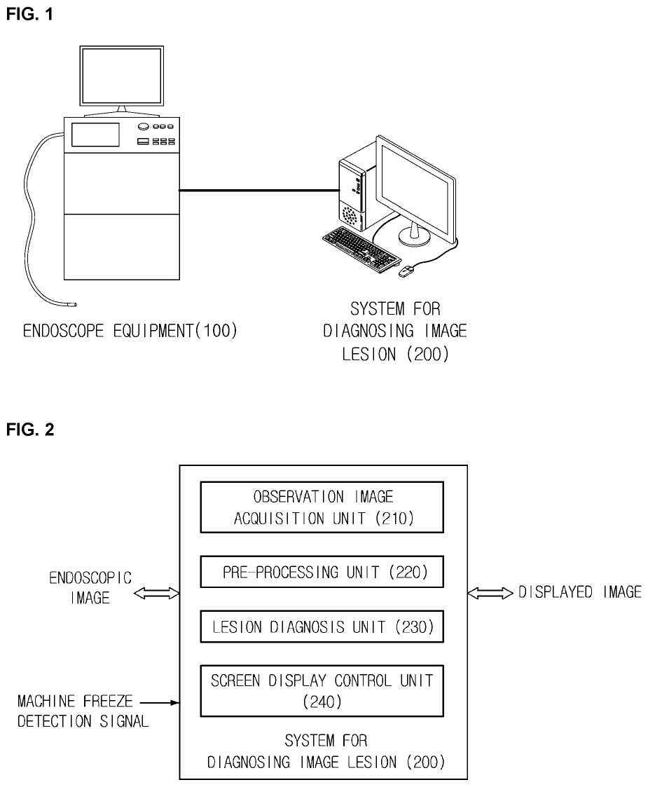

FIG. 1 is a diagram illustrating a peripheral configuration of a system 200 for diagnosing an image lesion according to an embodiment of the present invention.

The system 200 for diagnosing the image lesion according to an embodiment of the present invention can be implemented as an independent system or as a collection of program data (application program) installed in a computer system of a specialist (diagnostician) and executable in a main processor of the computer system. In some cases, it may be implemented and executed in the form of an application program executable in the main processor (control unit) of endoscopic equipment.

FIG. 1 illustrates the system 200 for diagnosing the image lesion installed in the computer system of the specialist, and, depending on the implementation method, the system 200 for diagnosing the image lesion automatically diagnoses and displays a degree of lesion on a freeze image transmitted from the endoscope equipment 100, or detects and displays a lesion area on a real-time endoscopic image, or detects the lesion area on the real-time endoscopic image and automatically diagnoses and displays the degree of lesion and/or infiltration depth for the detected lesion area.

For reference, the endoscope equipment 100 illustrated in FIG. 1 may be gastric endoscope equipment, small intestine endoscope equipment, and large intestine endoscope equipment. The endoscopic equipment 100 displays an endoscopic image obtained by an endoscope on a display unit. The endoscope equipment 100 and the computer system in which the system 200 for diagnosing the image lesion is installed are mutually connected through cables and image output terminals, so that the same endoscopic image displayed on the endoscope equipment 100 can be displayed on a display unit of the computer system of the specialist.

Meanwhile, the system 200 for diagnosing the image lesion according to an embodiment of the present invention, depending on the type of endoscope, may also automatically detect or diagnose the lesion area and the degree of lesion on one or more endoscopic images selected from among a gastric endoscope image, a small intestine endoscopy image, and a large intestine endoscopy image.

The system 200 for diagnosing the image lesion is further described below.

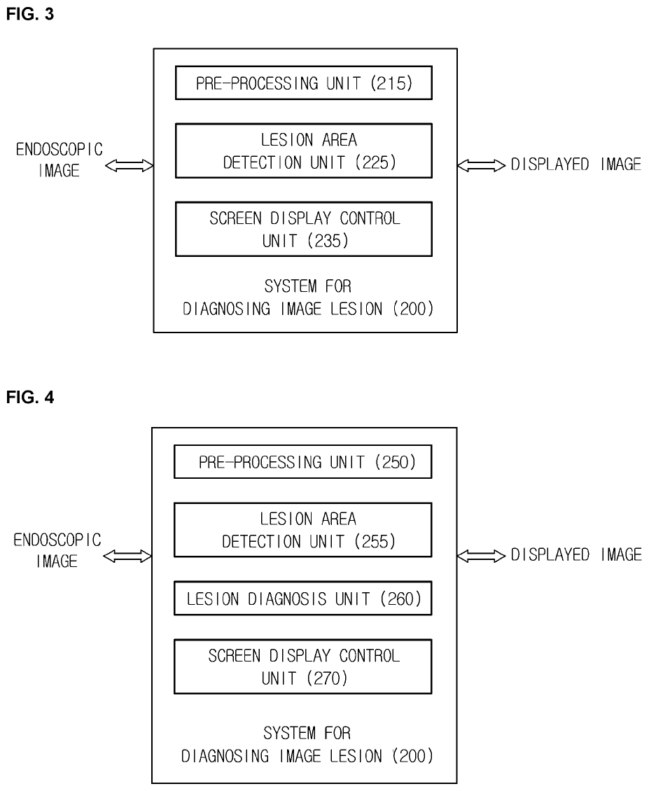

FIGS. 2 to 4 illustrate configuration diagrams of the system for diagnosing the image lesion according to the embodiments of the present invention, respectively. FIG. 2 illustrates the system for diagnosing the image lesion capable of automatically diagnosing and displaying the degree of lesion on the freeze image transmitted from the endoscopic equipment 100, FIG. 3 illustrates a system for automatically detecting and displaying the lesion area for the real-time endoscopic image, and FIG. 4 illustrates a system for automatically detecting the lesion area on the real-time endoscopic image and automatically diagnosing and displaying the degree of lesion and/or infiltration depth for the detected lesion area.

Referring first to FIG. 2, the system 200 for diagnosing the image lesion according to the first embodiment of the present invention includes

an observation image acquisition unit 210 configured to acquire an observation image from an endoscope image input from the endoscope equipment 100,

a pre-processing unit 220 configured to pre-process the acquired observation image,

a lesion diagnosis unit 230 configured to diagnose the degree of lesion on the pre-processed observation image using a pre-trained artificial neural network learning model for lesion diagnosis, and

a screen display control unit 240 configured to display and output a lesion diagnosis result.

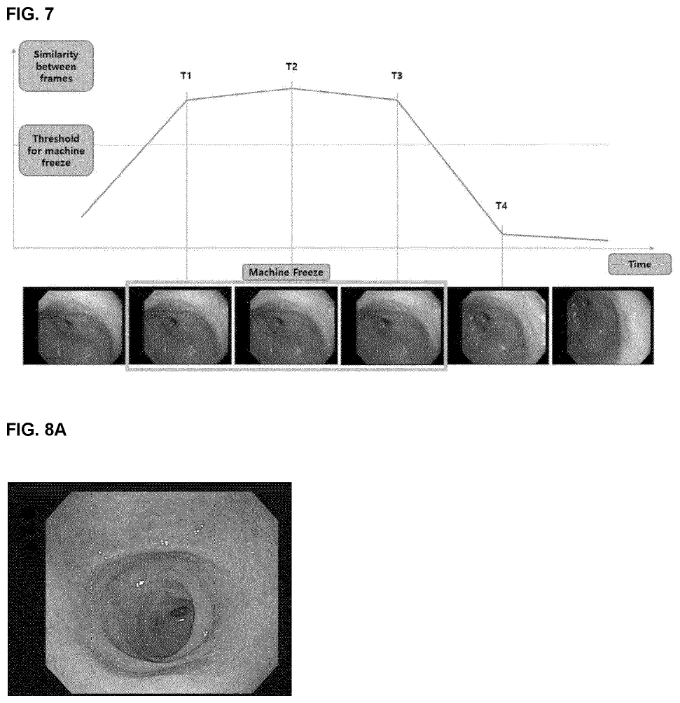

The observation image acquisition unit 210, as illustrated in FIG. 7, acquires (understood as capture) image frames (image frames at T1, T2, and T3) whose inter-frame similarity exceeds a predetermined threshold (i.e., recognized as machine freeze) among frames of the endoscopic image. When it is machine-frozen by a diagnostician performing endoscopic diagnosis on the side of the endoscope equipment 100, the frozen endoscopic image is temporarily stopped and displayed, and thus the inter-frame similarity at this time can be said to be very high. As such, when the inter-frame similarity exceeds the predetermined threshold, the system 200 for diagnosing the image lesion can recognize that an endoscopic image has been frozen on the side of the endoscopic equipment 100, and diagnose whether or not a lesion is present on the corresponding image.

In some cases, a machine freeze operation of an operator of the endoscopic equipment 100 may be detected and an endoscopic image may be captured in conjunction therewith to diagnose whether or not the lesion is present in the corresponding image.

That is, the observation image acquisition unit 210 may capture and acquire the endoscope image as the observation image when an electric signal generated according to the machine freeze operation of the operator of the endoscope equipment 100 is input. The electrical signal is preferably understood as a detection signal that detects that the operator of the endoscopic equipment 100 operates the equipment (operation of a handle or footrest of the endoscope equipment) in order to freeze the endoscopic image.

The pre-processing unit 220 removes unnecessary parts (noise), e.g., blood, text, biopsy instruments, etc., from an endoscopic image in frame units in order to diagnose the lesion. The pre-processing unit 220 may concurrently perform a pre-processing process of extracting a part marked as the lesion area by the specialist, etc. and smoothing an edge portion.

On the other hand, the lesion diagnosis unit 230 may detect the lesion area in the pre-processed observation image using a pre-trained artificial neural network learning model for lesion diagnosis, and then diagnose the degree of lesion for the detected lesion area.

The artificial neural network learning model for lesion diagnosis may be a network structure in which a convolution layer and a pooling layer are repeated between an input layer and a fully connected layer, as in a convolution neural network learning model, which is one of the artificial neural networks, and may be a structure disclosed in the patent application No. 10-2020-0007623 previously filed by the applicant of the present application, that is, a structure including a group of convolution layers and a group of deconvolution layers that process a convolution operation and a deconvolution operation in parallel in either of the pooling layer and the repeated convolution layer for noise mitigation, respectively, and including an add layer that combines feature maps that have passed through the group of convolutional layers and deconvolutional layers, respectively, into one and delivers them to a fully connected layer.

The artificial neural network learning model for lesion diagnosis is a model constructed by being pre-trained with endoscopic image data, in which the lesion area and/or degree of lesion are marked by the specialist, through a deep learning algorithm, and, in a diagnosis mode, automatically diagnoses the degree of lesion on a pre-processed observation image, or detects the lesion area in the pre-processed observation image and then diagnoses the degree of lesion for the detected lesion area.

For reference, in the artificial neural network learning model for lesion diagnosis, it is trained with a pair (x, y) of input x and one output y corresponding to the input. The input may be an image, and the output may be, for example, the degree of lesion.

In addition, each artificial neural network learning model used in the embodiment of the present invention can learn data augmented training data in order to construct a robust learning model. Types of data augmentation include left/right inversion, up/down inversion, rotation (−10° to +10°), and blur, and a ratio of learning of training data, validation, and test data can be adjusted to 6:2:2.

In addition, each artificial neural network learning model used in the embodiment of the present invention may use a modified DenseNet based convolutional neural network to which hyper-parameter tuning is applied.

Meanwhile, the lesion diagnosis unit 230 may include a pre-trained artificial neural network learning models for one or more lesions diagnosis in order to diagnose the degree of lesion on each of one or more endoscopic images among a gastric endoscope image, a small intestine endoscopy image, and a large intestine endoscopy image. Accordingly, the system 200 for diagnosing the image lesion may diagnose the lesion on the gastric endoscope image or the lesion on the large intestine endoscopy image. In some cases, it may further diagnose the lesion on the small intestine endoscopic image.

For reference, if the artificial neural network learning model for lesion diagnosis is a diagnostic model for the gastric endoscopic image, it is pre-trained to diagnose the lesion so that it can diagnose normal, low grade dysplasia (LGD), high grade dysplasia (HGD), early gastric cancer (EGC), and advanced gastric cancer (AGC) as the degrees of lesion, if it is a diagnostic model for the small intestine endoscopic image, it is pre-trained to diagnose the lesion so that it can diagnose bleeding, ulcers, vasodilation, and cancer tumors, and if it is a diagnostic model for the large intestine endoscopic image, it is pre-trained to diagnose the lesion so that it can diagnose non-neoplasm, tubular adenoma (TA), HGD, and cancer.

Hereinafter, the system 200 for diagnosing the image lesion according to another embodiment of the present invention will be further described with reference to FIG. 3. The system 200 for diagnosing the image lesion illustrated in FIG. 3 includes

a pre-processing unit 215 configured to pre-process the endoscopic image input from the endoscopic equipment 100,

a lesion area detection unit 225 configured to detect the lesion area in real time from the pre-processed endoscopic image frame using the pre-trained artificial neural network learning model for real-time lesion area detection, and

a screen display control unit 235 configured to display and output an endoscopic image frame in which the detected lesion area is marked.

The pre-processing unit 215 may recognize and remove blood, text, and biopsy instruments from the endoscopic image in frame units, and the lesion area detection unit 225 also includes a pre-trained artificial neural network learning models for one or more lesions detection in order to detect the lesion area for each of one or more endoscopic images among the gastric endoscope image, the small intestine endoscopy image, and the large intestine endoscopy image.

The artificial neural network learning model for lesion area detection is also a model that has previously learned training data using a deep learning algorithm called convolutional neural network (CNN), and the training data may be referred to as an endoscopic image in which the lesion area is marked by the specialist.

The system 200 for diagnosing the image lesion illustrated in FIG. 3 is a system that automatically detects the lesion area from the endoscopic image frame input in real time and displays the endoscopic image frame in which the detected lesion area is marked on a display unit. If the lesion area is automatically detected on the endoscopic image frame, an image frame in which the lesion area is marked is displayed on the display unit, and thus the diagnostician can concentrate on observing the corresponding image during the image frame in which the lesion area is marked is displayed. Depending on an implementation method, the endoscopic image frames in which the lesion area is marked may be separately stored and managed in an internal memory of the computer system.

As illustrated in FIG. 4, the system 200 for diagnosing the image lesion according to another embodiment of the present invention includes

a pre-processing unit 250 configured to pre-process an input endoscopic image,

a lesion area detection unit 255 configured to detect the lesion area in real time from the pre-processed endoscopic image frame using the pre-trained artificial neural network learning model for real-time lesion area detection,

a lesion diagnosis unit 260 configured to diagnose the degree of lesion for the detected lesion area using the pre-trained artificial neural network learning model for lesion diagnosis, and

a screen display control unit 270 configured to display and output the detected lesion area and the lesion diagnosis result.

In the system 200 for diagnosing the image lesion including these configurations, the pre-processing unit 250 recognizes and removes blood, text, and biopsy instruments from the endoscopic image frame.

Furthermore, the lesion area detection unit 255 may include the pre-trained artificial neural network learning model for one or more lesion areas detection in order to detect the lesion area for each of one or more endoscopic images among the gastric endoscope image, the small intestine endoscopy image, and the large intestine endoscopy image, and

the lesion diagnosis unit 260 may also include the pre-trained artificial neural network learning model for one or more lesions diagnosis in order to diagnose the degree of lesion for each of one or more endoscopic images among the gastric endoscope image, the small intestine endoscopy image, and the large intestine endoscopy image.

Each of the imaging lesion diagnosis systems 200 described for each embodiment above may further include a technical configuration for notifying a diagnostician or specialist of the fact through an alarm when the lesion area is detected. In addition, if each system 200 for diagnosing the image lesion is a system for diagnosing the lesion on the large intestine endoscopy image, the lesion diagnosis unit may diagnose and display the infiltration depth.

Hereinafter, the operation of the system 200 for diagnosing the image lesion described above will be described in more detail, but the operation of diagnosing the gastric endoscopic image will be described below.

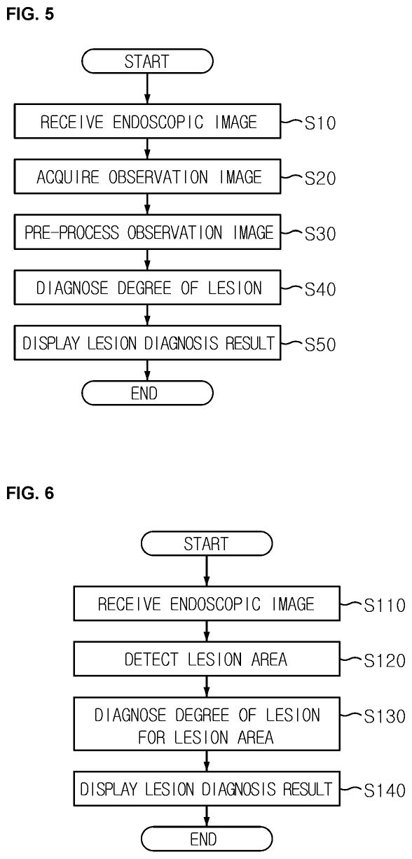

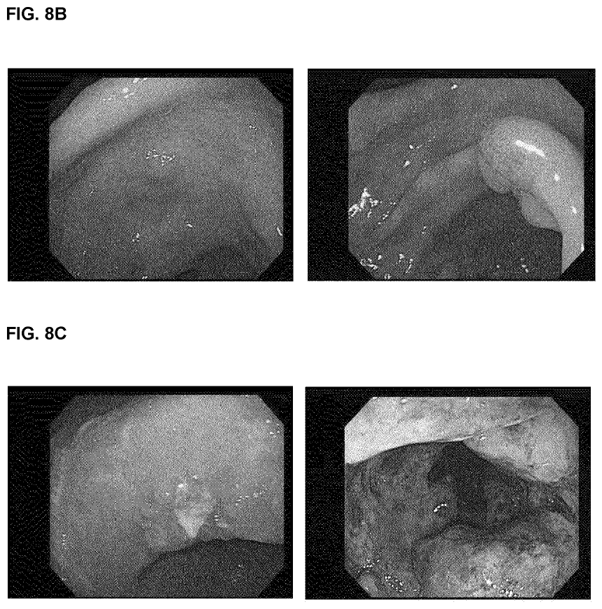

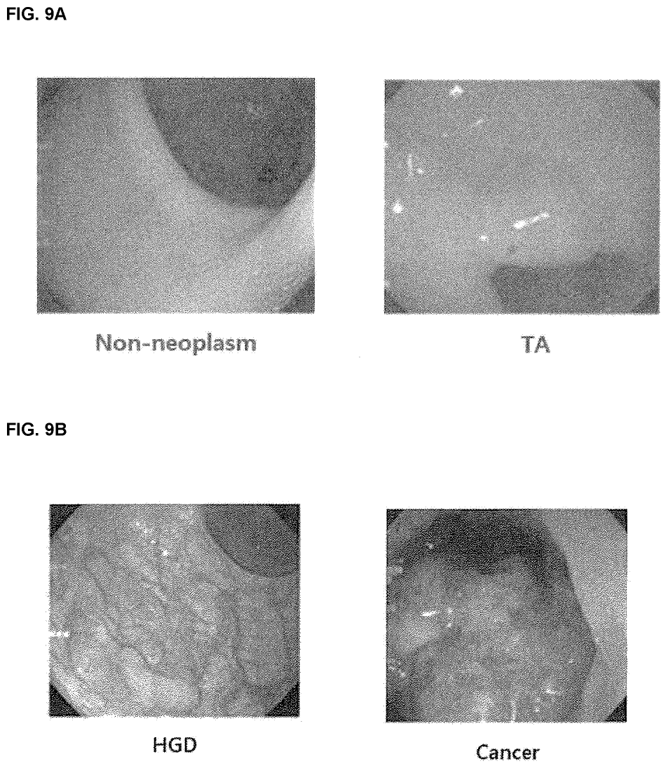

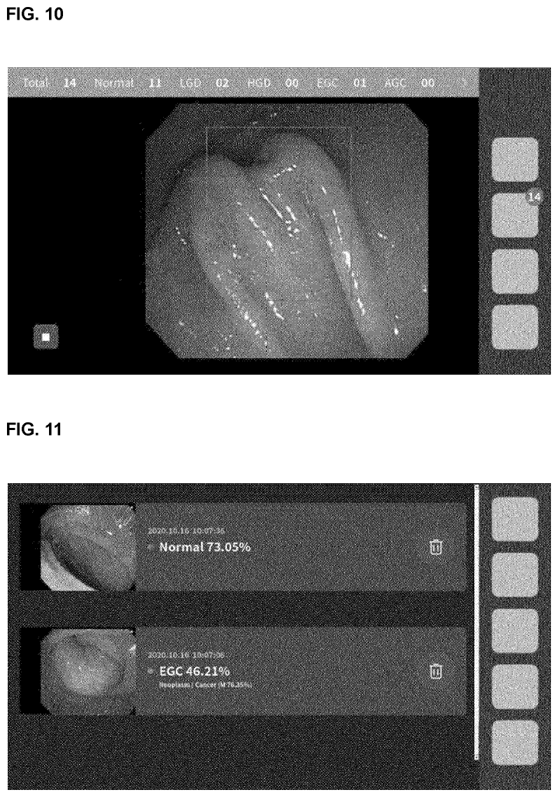

FIG. 5 illustrates an operational flow diagram of the system 200 for diagnosing the image lesion according to an embodiment of the present invention, FIG. 7 illustrates a diagram for describing observation image acquisition according to an embodiment of the present invention, FIGS. 8A to 9B illustrate exemplary diagrams for diagnosing the degree of lesions in endoscopic images according to embodiments of the present invention, and FIGS. 10 and 11 are exemplary diagrams of lesion diagnosis screens according to an embodiment of the present invention.

First, prior to diagnosing the lesion on the endoscopic image, the system 200 for diagnosing the image lesion should train the artificial neural network learning model for lesion diagnosis through a learning mode.

For example, on the gastroscopic image, the specialist marks the lesion area and inputs information on the degree of lesion. In this way, a plurality of endoscopic image frames in which the lesion area and lesion degree information are marked or inputted are delivered to the artificial neural network learning model for lesion diagnosis having a deep neural network structure according to a specialist's command.

Accordingly, the artificial neural network learning model for lesion diagnosis learns training data, that is, the features of the image in which the lesion area is marked in the gastroscopic image, goes through testing and verification steps, and ends learning of a model for predicting any one of normal/LGD/HGD/EGC/AGC as the degree of lesion on the gastric endoscopic image.

In this way, if the artificial neural network learning model for lesion diagnosis is trained, the degree of lesion on the gastric endoscopic image can be diagnosed based on this learning model.

Referring to FIG. 5, first, the gastroscopic endoscopic image obtained through the endoscope is displayed on the display unit of the endoscope equipment 100, and is received by the specialist's PC installed with the system 200 for diagnosing the image lesion (step S10) and displayed on the display unit.

Therefore, the observation image acquisition unit 210 of the system 200 for diagnosing the image lesion may acquire an observation image from the received endoscopic image (step S20).

As illustrated in FIG. 7, in the method for obtaining the observation image, image frames whose inter-frame similarity exceeds a predetermined threshold among the frames of the endoscopic image, for example, image frames at time points T1, T2, and T3 illustrated in FIG. 7 may be acquired as the observation image.

As another method, the observation image acquisition unit 210 may capture and acquire the endoscope image as the observation image when an electrical signal generated according to a machine freeze operation of an operator of the endoscope equipment is input.

The acquired observation image is pre-processed by the pre-processing unit 220 and delivered to the lesion diagnosis unit 230. The pre-processing unit 220 for removing an unnecessary area and object for diagnosing the lesion may be designed differently depending on the type of diagnostic images (gastric endoscope, small intestine endoscope, large intestine endoscope).

For example, pre-processing can be performed so that images of text, auxiliary diagnostic equipment, blood, and organs other than the observation target, etc., which are unnecessary for diagnosis of the lesion, may be removed as necessary.

When preprocessing is completed for the observation image and the observation image is delivered to the lesion diagnosis unit 230, the lesion diagnosis unit 230 diagnoses the degree of lesion on the pre-processed observation image using the pre-trained artificial neural network learning model for lesion diagnosis (step S40).

After that, the screen display control unit 240 displays and outputs the lesion diagnosis result delivered from the lesion diagnosis unit 230 (step S50). As illustrated in FIGS. 8A to 8C, diagnosis of the degrees of lesion on the gastric endoscopic image can be classified into normal (FIG. 8A), LGD/HGD (FIG. 8B), and EGC/AGC (FIG. 8C).

If the artificial neural network learning model for lesion diagnosis is a pre-trained learning model in order to diagnose the degree of lesion on the large intestine endoscopy image, the lesion diagnosis unit 230 can automatically diagnose and display four types of lesions in the large intestine endoscopy image as illustrated in FIGS. 9A and 9B.

As described above, the system 200 for diagnosing the image lesion according to a first embodiment of the present invention has an advantage capable of diagnosing the lesion by automatically recognizing the machine-frozen image in the endoscopic equipment 100 and obtaining the image as the observation image, and furthermore, has the advantage capable of obtaining objective and highly reliable diagnosis results regardless of the experience, ability, and proficiency of the specialist because the degree of the lesion is automatically diagnosed using the pre-trained artificial neural network learning model for lesion diagnosis on an acquired observation image, that is, the image frozen in the endoscopic equipment 100 and the result is displayed.

In addition, since the system 200 for diagnosing the image lesion according to the first embodiment of the present invention can construct the lesion diagnosis unit 230 so that the degree of lesion can be automatically diagnosed by acquiring the small intestine endoscopic image the an observation image, there is convenience capable of automatically diagnosing the degree of lesion for the gastric endoscope, large intestine endoscope, and small intestine endoscope according to an operation mode (which is set to gastric endoscope diagnosis mode, . . . , internal organ endoscopy diagnosis mode) with only one system construction.

Hereinafter, the operation of the system 200 for diagnosing the image lesion according to a second embodiment of the present invention illustrated in FIG. 3 will be further described.

Prior to detecting the lesion area in real time on the endoscopic image, the system 200 for diagnosing the image lesion illustrated in FIG. 3 should train the artificial neural network learning model for lesion area detection through the learning mode.

For example, on the gastroscopic image, the specialist marks the lesion area. In this way, the plurality of endoscopic image frames in which the lesion area is marked are delivered to the artificial neural network learning model for lesion area detection having a deep neural network structure according to the specialist's command.

Accordingly, the artificial neural network learning model for lesion area detection learns the features of the image in which the lesion area is marked, goes through testing and verification steps, and ends learning of a model for detecting the lesion area on the gastric endoscopic image.

In this way, if the artificial neural network learning model for lesion area detection is trained, the lesion area on the gastric endoscopic image can be automatically detected based on this learning model.

First, the gastroscopic endoscopic image obtained through the endoscope is displayed on the display unit of the endoscope equipment 100, and is received by the specialist's PC installed with the system 200 for diagnosing the image lesion (step S10) and displayed on the display unit.

Accordingly, the pre-processing unit 215 of the system 200 for diagnosing the image lesion pre-processes the received endoscopic image. As described above, the pre-processing unit 215 performs pre-processing so that images of the area and object, such as text, auxiliary diagnostic devices, blood, organs other than the observation target, etc., which are unnecessary for detecting the lesion area, may be removed as necessary.

The pre-processed gastroscopic endoscopic image is delivered to the lesion area detection unit 225, and the lesion area detection unit 225 detects the lesion area in real time from the pre-processed gastroscopic endoscope image frame using the pre-trained artificial neural network learning model for real-time lesion area detection. If the lesion area is detected, the lesion area detection unit 225 delivers coordinate information for displaying the lesion area to the screen display control unit 235. Accordingly, the screen display control unit 235 displays and outputs gastric endoscope image frames in which the lesion area (square box) is marked as illustrated in FIG. 10.

That is, the system 200 for diagnosing the image lesion according to the second embodiment of the present invention automatically displays the gastric endoscopic image in which the lesion area is marked (or with simultaneous alarm output) when the lesion area is detected in real time on the gastric endoscopic image, so that the diagnostician such as the specialist may additionally diagnose the degree of lesion by intensively observing the image frame in which the lesion area is marked, or readjust the position of the endoscope in order to check an image of the surroundings from which the image, in which the lesion area is marked, is obtained.

The lesion area detection unit 225 described above, by including the pre-trained artificial neural network learning model for one or more lesion areas detection in order to detect the lesion area for each of one or more endoscopic images among the gastric endoscope image, the small intestine endoscopy image, and the large intestine endoscopy image, also has the advantage capable of automatically detecting and displaying the lesion area for gastric endoscope, large intestine endoscope, and small intestine endoscope according to an operation mode (which is set to gastric endoscope diagnosis mode, . . . , internal organ endoscopy diagnosis mode) with only one system construction.

Hereinafter, the operation of the system 200 for diagnosing the image lesion according to a third embodiment of the present invention illustrated in FIG. 4 will be further described with reference to FIG. 6.

First, prior to detecting the lesion area in real time for the endoscopic image, the system 200 for diagnosing the image lesion illustrated in FIG. 4 should train the artificial neural network learning model for lesion area detection through the learning mode. Since the learning process of the artificial neural network learning model for lesion area detection has been described above, it will be omitted below.

In addition to training the artificial neural network learning model for lesion area detection, the system 200 for diagnosing the image lesion according to the third embodiment should train the artificial neural network learning model for lesion diagnosis for diagnosing the degree of lesion. Since the learning process of such an artificial neural network learning model for lesion diagnosis has already been described in FIG. 5, it will be omitted below.

If the artificial neural network learning model for lesion area detection and the artificial neural network learning model for lesion diagnosis are trained, the lesion area and the degree of lesion on the gastric endoscopic image, the large intestine endoscopy image, and the small intestine endoscopy image can be automatically detected based on these learning models.

Referring to FIG. 6, first, the gastroscopic endoscopic image obtained through the endoscope is displayed on the display unit of the endoscope equipment 100, and is received in real time by the specialist's PC installed with the system 200 for diagnosing the image lesion (step S110) and displayed on the display unit.

Accordingly, the pre-processing unit 250 of the system 200 for diagnosing the image lesion pre-processes the received endoscopic image. As described above, the pre-processing unit 215 performs pre-processing so that images of the area and object, such as text, auxiliary diagnostic devices, blood, organs other than an observation target, etc., which are unnecessary for detecting the lesion area, may be removed as necessary.

The pre-processed gastroscopic endoscopic image is delivered to the lesion area detection unit 255, and the lesion area detection unit 255 detects the lesion area in real time from the pre-processed gastroscopic endoscope image frame using the pre-trained artificial neural network learning model for real-time lesion area detection (step S120). If the lesion area is detected, the lesion area detection unit 255 delivers coordinate information for displaying the lesion area to the screen display control unit 270, and delivers the detected lesion area image to the lesion diagnosis unit 260.

The lesion diagnosis unit 260 diagnoses the degree of lesion for the detected lesion area using the pre-trained artificial neural network learning model for lesion diagnosis (step S130).

After that, the screen display control unit 270 displays and outputs the lesion diagnosis result delivered from the lesion diagnosis unit 260 (step S140).

Accordingly, as illustrated in FIG. 10 or 11, on the display unit, the lesion area detected for the endoscopic image may be marked and displayed or the degree of lesion may be displayed together, and the possibility of the degree of the diagnosed lesion may be displayed together.

The system 200 for diagnosing the image lesion according to the third embodiment of the present invention also has the advantage capable of obtaining objective and highly reliable diagnosis results regardless of the experience, ability, and proficiency of the specialist by automatically detecting the lesion area in real time on the endoscopic image and automatically diagnosing the degree of lesion for the detected lesion area.

In addition, the present invention, by including the pre-trained artificial neural network learning model for one or more lesion areas detection and the pre-trained artificial neural network learning model for one or more lesion diagnoses in order to detect the lesion area for each of one or more endoscopic images among the gastric endoscope image, the small intestine endoscopy image, and the large intestine endoscopy image, has the advantage capable of automatically diagnosing the degree of lesion by automatically detecting the lesion area for gastric endoscope, large intestine endoscope, and small intestine endoscope according to the operation mode (which is set to gastric endoscope diagnosis mode, . . . , internal organ endoscopy diagnosis mode) with only one system construction.

Meanwhile, in the above embodiments, the system for detecting a lesion or diagnosing the degree of lesion in the endoscopic image by installing the system 200 for diagnosing the image lesion in the specialist's PC has been described, but the system 200 for diagnosing the image lesion described above may be installed in the endoscopic equipment 100 or may be implemented as an embedded system to be executed in a main processor of the endoscopic equipment 100.

One endoscopic equipment 100 may be constructed, as illustrated in FIG. 2, by further including, for example, in the system for diagnosing the image lesion (preferably understood as endoscope equipment) including the endoscope including an insertion unit inserted into a human body and an image sensing unit which is positioned within the insertion unit and senses light reflected from the human body to generate an endoscope image signal, the image signal processing unit for processing an endoscopic image signal captured by the endoscope into a displayable endoscopic image, and the display unit for displaying the endoscopic image,

the observation image acquisition unit 210 configured to acquire an observation image from the endoscopic image,

the pre-processing unit 220 configured to pre-process an acquired observation image,

the lesion diagnosis unit 230 configured to diagnose the degree of lesion on the pre-processed observation image using the pre-trained artificial neural network learning model for lesion diagnosis, and

the screen display control unit 240 configured to display and output a lesion diagnosis result.

In addition, one endoscopic equipment 100 may be constructed, as illustrated in FIG. 4, by further including, in the system for diagnosing the image lesion (preferably understood as endoscope equipment) including the endoscope including an insertion unit inserted into a human body and an image sensing unit which is positioned within the insertion unit and senses light reflected from the human body to generate an endoscope image signal, the image signal processing unit for processing an endoscopic image signal captured by the endoscope into a displayable endoscopic image, and the display unit for displaying the endoscopic image,

the pre-processing unit 250 configured to pre-process the endoscopic image,

the lesion area detection unit 255 configured to detect the lesion area in real time from the pre-processed endoscopic image frame using the pre-trained artificial neural network learning model for real-time lesion area detection,

the lesion diagnosis unit 260 configured to diagnose the degree of lesion for the detected lesion area using the pre-trained artificial neural network learning model for lesion diagnosis, and

the screen display control unit 270 configured to display and output the detected lesion area and the lesion diagnosis result.

For reference, the lesion diagnosis unit 230 of the system 200 for diagnosing the image lesion according to the embodiment of the present invention may make a diagnosis per captured image when multiple captured images are acquired for the same lesion, but it is most preferable to make a diagnosis using an average value. The lesion diagnosing unit 230 may be configured to make a diagnosis based on the most serious severity or may make a diagnosis in consideration of the frequency.

Based on the description of the above embodiments, a person skilled in the art can clearly understand that the present invention can be achieved through a combination of software and hardware or can be achieved only by hardware. Objects of the technical solution of the present invention or parts contributing to the prior art thereof may be implemented in the form of program instructions that can be executed through various computer components and recorded on a machine-readable recording medium. The machine-readable recording medium may include program instructions, data files, data structures, etc. alone or in combination. The program instructions recorded on the machine-readable recording medium may be those specially designed and configured for the present invention or may be known to and usable by a person skilled in the art of computer software. Examples of the program instructions include not only machine language codes such as those produced by compilers, but also high-level language codes that can be executed by a computer using an interpreter, etc. The hardware device may be configured to operate as one or more software modules in order to perform processing according to the present invention, and vice versa. The hardware device may include a processor such as a CPU or GPU coupled to a memory such as ROM/RAM for storing the program instructions and configured to execute the instructions stored in the memory, and may include a communication unit capable of transmitting and receiving signals to and from an external device. In addition, the hardware device may include a keyboard, mouse, and other external input devices for receiving commands written by developers.

Although the present invention has been described by specific details such as specific components and limited embodiments and drawings, these are provided only to help a more general understanding of the present invention, and the present invention is not limited to the embodiments described above. A person skilled in the art to which the present invention pertains may make various modifications and variations from these descriptions. Accordingly, the spirit of the present invention should not be limited to and should not be determined by the embodiments described above, and it will be said that not only the claims described later, but also all things modified to be equal to or equivalent to these claims belong to the scope of the spirit of the present invention.

1. A system for diagnosing an image lesion, the system comprising:
an observation image acquisition unit configured to acquire an observation image from an input endoscopic image;
a pre-processing unit configured to pre-process an acquired observation image;
a lesion diagnosis unit configured to diagnose a degree of lesion on the pre-processed observation image using a pre-trained artificial neural network learning model for lesion diagnosis; and
a screen display control unit configured to display and output a lesion diagnosis result.

2. The system of claim 1, wherein the observation image acquisition unit is configured to acquire, as the observation image, image frames whose inter-frame similarity exceeds a predetermined threshold among frames of the endoscopic image.

3. The system of claim 1, wherein the observation image acquisition unit is configured to capture and acquire the endoscopic image as an observation image when an electric signal generated according to a machine freeze operation of an endoscope equipment operator is input.

4. The system of claim 1, wherein the lesion diagnosis unit includes a pre-trained artificial neural network learning model for one or more lesions diagnosis in order to diagnose a degree of lesion for each of one or more endoscopic images among a gastric endoscope image, a small intestine endoscopy image, and a large intestine endoscopy image.

5. The system of claim 1, wherein the lesion diagnosis unit is configured to detect a lesion area in the pre-processed observation image using the pre-trained artificial neural network learning model for lesion diagnosis, and then diagnoses the degree of lesion for the detected lesion area.

6. The system of claim 1, wherein the artificial neural network learning mod& for lesion diagnosis is configured to diagnose normal, low-grade dysplasia, high-grade dysplasia, early gastric cancer, and advanced gastric cancer on a gastric endoscopic image.

7. A system for diagnosing an image lesion, the system comprising:
a pre-processing unit configured to pre-process an input endoscopic image;
a lesion area detection unit configured to detect a lesion area in real time from the pre-processed endoscopic image frame using a pre-trained artificial neural network learning model for real-time lesion area detection; and
a screen display control unit configured to display and output an endoscopic image frame in which the detected lesion area is marked.

8. The system of claim 7, wherein the pre-processing unit is configured to recognize and remove blood, text, and biopsy instruments from the endoscopic image in frame units.

9. The system of claim 7, wherein the lesion area detection unit includes a pre-trained artificial neural network learning model for one or more lesion areas detection in order to detect a lesion area for each of one or more endoscopic images among a gastric endoscope image, a small intestine endoscopy image, and a large intestine endoscopy image.

10. A system for diagnosing an image lesion, the system comprising:
a pre-processing unit configured to pre-process an input endoscopic image;
a lesion area detection unit configured to detect a lesion area in real time from the pre-processed endoscopic image frame using a pre-trained artificial neural network learning model for real-time lesion area detection;
a lesion diagnosis unit configured to diagnose a degree of lesion for the detected lesion area using a pre-trained artificial neural network learning model for lesion diagnosis; and
a screen display control unit configured to display and output the detected lesion area and a lesion diagnosis result.

11. The system of claim 10, wherein the pre-processing unit is configured to recognize and remove blood, text, and biopsy instruments from an endoscopic image frame.

12. The system of claim 10, wherein the lesion area detection unit includes a pre-trained artificial neural network learning model for one or more lesion areas detection in order to detect a lesion area for each of one or more endoscopic images among a gastric endoscope image, a small intestine endoscopy image, and a large intestine endoscopy image.

13. The system of claim 10, wherein the lesion diagnosis unit includes a pre-trained artificial neural network learning model for one or more lesions diagnosis in order to diagnose a degree of lesion for each of one or more endoscopic images among a gastric endoscope image, a small intestine endoscopy image, and a large intestine endoscopy image.

14. A system for diagnosing an image lesion that includes an endoscope including an insertion unit inserted into a human body and an image sensing unit which is positioned within the insertion unit and senses light reflected from the human body to generate an endoscope image signal, an image signal processing unit for processing an endoscopic image signal captured by the endoscope into a displayable endoscopic image, and a display unit for displaying the endoscopic image, the system comprising:
an observation image acquisition unit configured to acquire an observation image from the endoscopic image;
a pre-processing unit configured to pre-process an acquired observation image;
a lesion diagnosis unit configured to diagnose a degree of lesion on the pre-processed observation image using a pre-trained artificial neural network learning model for lesion diagnosis; and
a screen display control unit configured to display and outputs a lesion diagnosis result.

15. A system for diagnosing an image lesion that includes an endoscope including an insertion unit inserted into a human body and an image sensing unit which is positioned within the insertion unit and senses light reflected from the human body to generate an endoscope image signal, an image signal processing unit for processing an endoscopic image signal captured by the endoscope into a displayable endoscopic image, and a display unit for displaying the endoscopic image, the system comprising:
a pre-processing unit configured to pre-process the endoscopic image;
a lesion area detection unit configured to detect a lesion area in real time from the pre-processed endoscopic image frame using a pre-trained artificial neural network learning model for real-time lesion area detection;
a lesion diagnosis unit configured to diagnose a degree of lesion for the detected lesion area using a pre-trained artificial neural network learning model for lesion diagnosis; and
a screen display control unit configured to display and output a detected lesion area and a lesion diagnosis result.

16. The system of claim 2, wherein the lesion diagnosis unit is configured to detect a lesion area in the pre-processed observation image using the pre-trained artificial neural network learning model for lesion diagnosis, and then diagnoses the degree of lesion for the detected lesion area.

17. The system of claim 3, wherein the lesion diagnosis unit is configured to detect a lesion area in the pre-processed observation image using the pre-trained artificial neural network learning model for lesion diagnosis, and then diagnoses the degree of lesion for the detected lesion area.

18. The system of claim 4, wherein the lesion diagnosis unit is configured to detect a lesion area in the pre-processed observation image using the pre-trained artificial neural network learning model for lesion diagnosis, and then diagnoses the degree of lesion for the detected lesion area.
